(19) United States
(12) Patent Application Publication
(10) Pub. No.: US 2011/0171668 A1
(43) Pub. Date: Jul. 14, 2011

(54) Determination Of Equilibrium Constants In Solution By Multi-Step Kinetics

(76) Inventor: Hans-Heinrich Trutnau, Eltvile (DE)

(21) Appl. No.: 12/954,647

(22) Filed: Nov. 25, 2010

Related U.S. Application Data
(63) Continuation-in-part of application No. PCT/EP2009/059041, filed on Jul. 15, 2009.

(30) Foreign Application Priority Data
Sep. 9, 2008 (DE) DE102008044506

(51) Int. Cl. G01N 33/53; G01N 33/00; G06F 19/00

(52) U.S. Cl. 435/7.92; 436/34; 702/19

(57) ABSTRACT

The invention relates to a method for determining a first equilibrium constant of a first equilibrium reaction via MSK analysis of a second equilibrium reaction, whereby the first and the second equilibrium reaction share at least one reactant.

### FIELD OF THE INVENTION

The invention relates to a method by which a first equilibrium constant of a first equilibrium reaction currently taking place is determined, whereby for determining the first equilibrium constant MSK kinetics of a second equilibrium reaction is determined.

In other words, the invention relates to a method for determining equilibrium constants in solution via Multi-Step Kinetics (MSK), whereby this determination is executed indirectly. The invention is applied during the determination of (e.g. dissociation or association) equilibrium constants of reversible equilibrium reactions, the determination being executed by biochemical laboratories, and in particular for determining the corresponding constants of equilibrium reactions which precede the actual analysis (e.g. using an affinity biosensor).

### BACKGROUND

In the biochemical life sciences, a variety of vital activities (e.g. signaling pathways etc.) are examined.

Signaling pathways, metabolic pathways and other important biochemical control loops comprise a variety of substances such as proteins, nucleic acids, carbohydrates, and other components which can interact with each other.

These interactions and activities usually are performed in the form of reversible equilibrium reactions (between two or more starting substances under formation of one or more products), which are characterized by equilibrium constants, which in turn define the specific concentration ratio between starting substances and products. In addition to the term “starting substance”, its synonym “educt” will also be used in the following.

For example, complexes consisting of two or more substances can form, which also can dissociate again. As a measure indicating whether a dissociation equilibrium reaction (hereinafter also called dissociation) in equilibrium rather lies on the side of the complex or on that of the starting substances, the so-called dissociation equilibrium constant K_{D} is used. The equilibrium of an association reaction (hereinafter also referred to as association) is described by the so-called association constant K_{A}. K_{D} and K_{A} respectively are equilibrium constants of equilibrium reactions. Since these equilibrium constants allow to make statements about the tendency of substances to build complexes with other substances and thus for example to achieve a biochemically and/or pharmacologically relevant reaction, the determination of equilibrium constants is of great biological, pharmaceutical and medical interest.

At equilibrium state, the ratio of the product of the concentrations of products and the product of the concentrations of the starting substances is constant. Thus, the concentrations are constant. The concentration of the reactants in equilibrium state is called equilibrium concentration.

Although forward and reverse reaction occur steadily, which means that the starting substances are transformed into products and the products are in turn transformed into starting substances, at equilibrium the net concentrations of starting substances and products do not change. This is because in equilibrium the speed of the forward reaction and of the reverse reaction are exactly the same, which means that per unit of time as much starting substance reacts into product as product is transformed back into starting substance.

These constants, i.e., the equilibrium constants of the individual equilibrium reactions, can be determined and characterized by different methods both kinetically (based on their temporal course to their equilibrium, i.e., based on the temporal course of the reaction until equilibrium has been reached) and thermodynamically (based on their final equilibrium state, i.e., based on the concentrations of starting substance(s), product(s) and corresponding stoichiometric factors), this being often subsumed under the term Biomolecular Interaction Analysis (BIA).

The following approaches for determining the equilibrium constant of a reaction are known in the art:

The determination of, for example, the so-called dissociation equilibrium constant (K_{D}) or the reciprocal association equilibrium constant (in short “affinity constant” K_{A}) of a reversible equilibrium reaction (e.g. between two main starting substances called ligand “L” and analyte “A”, under reversible formation of the complex “LA”, in short: L+A⇄LA), is the subject of daily laboratory practice in biochemistry and can, for example, be carried out by means of enzyme-linked immunosorbent assay (ELISA) or radio-immunoassay (RIA). The already older, conventional determination of such constants, based among others on ELISA or RIA, is often elaborate and returns for various reasons only approximated values, as for example in ELISA the determination is carried out (a) not in solution but on a solid phase (b), and in addition is carried out not or only coarsely time resolved (i.e., kinetically), but rather is often carried out solely based on the final equilibrium state (i.e., thermodynamically). In addition, RIA is known to be afflicted with disposal-related problems, as, for example, in RIA radioactive isotopes are used.

Such a direct analysis of the reaction (e.g. via ELISA or RIA) is disclosed to have been executed also by means of MSK, which has been placed under protection by EP 1 259 810 B1. It should be noted here that a MSK analysis can be performed with regard to the determination of the reaction signal by using biosensors as of EP 1 259 810 B1, but also by using other, e.g. fluorescence- or radioactivity-based, approaches for determining the reaction signal. In particular, MSK analyses can be based on ELISA and RIA assays.

By using an affinity biosensor, L is immobilized on the sensor surface of the biosensor. A is sequentially, at varying concentrations, brought in contact with L on the sensor surface, whereby the sensor surface is not regenerated, i.e., is not set to “zero” between the sequential analyte contacts, whereby the formation rate of LA is recorded over the time and finally the recorded formation rate curves are evaluated in respect to kinetics and equilibrium constant. This direct assay based on Multi-Step Kinetics (MSK) is prior art—as well as a corresponding traditional sequential (SEQ) analysis based on biosensors (such as e.g. Biacore instruments, now distributed by GE Healthcare, Uppsala, Sweden): SEQ implies that the contacting or the analysis of each single analyte solution is followed by a regeneration step and a washing step, for bringing the biosensor signal back to zero, i.e., the so-called baseline, before performing the analysis of the next analyte solution, a procedure which is very costly in respect to time and material and has other disadvantages. Common to these biosensor techniques (as opposed to ELISA or RIA) is the advantage that they can track the time course of the reaction (i.e., its kinetics) in high resolution, and that thus the equilibrium constant of the reaction can be determined not only thermodynamically, but also kinetically. However, it is considered as a disadvantage that even based on this kind of (direct) biosensor assay, the reaction (because L is immobilized, i.e., in SEQ and MSK) is tracked on a solid phase and not in solution.

A special case of determining equilibrium constants is determining the inhibition constant (K_{I}), by which the reversible inhibition of an analyte (A) by an inhibitor (I) in equilibrium is quantitatively described via the law of mass action (LMA), which will be explained below in greater detail. By its very nature K_{I} is nothing other than K_{D}, whereby I is used instead of L. Therefore I could be immobilized on the sensor surface, could be (still directly) brought in contact with A and used for determining K_{I}—which, however, is (a) not always practically feasible, when, for example, a multitude of inhibitors shall be examined in respect to their effect on A and therefore for each inhibitor a new, expensive sensor surface would have to be prepared, and which (b) would obviously further on imply that the determination would be executed not in solution, but (directly) on a solid phase. Also, an immobilization of A for the direct determination of I is often not desirable, because (a) I usually consists of small molecules that can cause problems regarding the strength of the measured binding signal due to their small size, and (b) the determination would still be executed not in solution, but on a solid phase.

Therefore, it was suggested in the art in various ways (see quotes from Karlsson and Nieba) to run the inhibition reactions between A and I prior to the actual biosensor measurements separately and in separate reaction solutions in order to analyze the reached inhibition-equilibrium of the respective reaction solution with an appropriate L on the sensor surface (also called inhibition in solution assay, ISA) afterwards.

In contrast to the direct assays described previously, ISA is an indirect assay since, based on the actual measurement, inferences are made backward on the preceding reaction. To aptly summarize the prior art, ISA means that (firstly) for determining K_{I} of a specific A/I starting substance pair, multiple (>5) solutions of mixtures of the starting substances are prepared, each solution comprising equal analyte starting concentration c₀(A), but variable inhibitor starting concentration, c₀(I), and that thus the reaction of A and I under formation of analyte-inhibitor complex AI is initiated (whereby, in case of a high excess of inhibitor, the analyte may be complexed almost completely!), and that (secondly) the respectively reversible reaction between A and I in each solution has reacted to completion (whereby in each solution, starting from the respective starting concentrations of A and I, an individual stationary equilibrium has been reached with the equilibrium concentrations of A, c_{eq}(A), and c_{eq}(I) and AI, c_{eq}(AI), which do not change any more in the respective reaction solutions) and that (thirdly) the reaction solutions are subsequently analyzed in a SEQ approach against L on the sensor in order to infer the K_{I} value of the preceding reactions. When performing said analyses of preceding equilibria, different (but partly elaborate, partly error prone) approaches are and have been used to infer, from the measuring values, either directly or via the with limitations correctly determined (free) equilibrium concentration of A, c_{eq}(A), the K_{I}-value. A free concentration of a reactant of an association or dissociation reaction is its solvent concentration in uncomplexed form. This needs to be explained by referring to the underlying LMA according to which the equilibrium concentrations and their mathematical ratio to each other provide for the K_{I} value as follows:

$${K}_{1} = \frac{{c}_{eq} ( A ) \times {c}_{eq} ( I )}{{c}_{eq} ( AI )}$$

However (as has been mentioned previously and as will be demonstrated below by means of practice examples), with total complexation and inhibition of the analyte, said analyte's c_{eq}(A) is practically zero, the K_{I} value can no longer or only with very large error value be determined from such a singular measurement, which (as set out in the following publications discussed by Karlsson and Nieba) necessitates extremely complex compensation evaluation procedures (which are probably the reason for the low usage frequency of this approach and which are rendered unnecessary by the method of the present invention).

Karlsson (1994 and 2000) discloses various types of inhibition in solution assays, all of which being characterized by the following disadvantages:
(a) SEQ approach comprising regeneration/washing phases between the analyte contacting steps;
(b) constant analyte starting concentration c₀(A), which can under some circumstances, when the inhibitor starting concentration c₀(I) is high, lead to an almost complete inhibition of A and which consequently generates a c_{eq}(A) which is practically not measurable;
(c) extremely high, but actually unnecessary immobilization density of L (which causes mass transport limitation);
(d) by drawing a simple regression line through the start of an at least weakly bent curve, distorted and merely relative initial rates (which are insufficient as an absolute measure of c_{eq}(A)) are obtained, which in addition are corrected multiple times; in one case uncorrected values turned out to be in better agreement with values having been obtained by using alternative methods than the corrected values;
(e) mathematically extremely complex evaluation procedures, which will rarely be followed by biologists and biochemists of current life science laboratories;
(f) erroneous information given in the publications, some of which being difficult or impossible to understand (for example, positive rather than negative exponents given in data values relating to concentration data).

Nieba (1996) discloses a so-called ‘Competition Assay’ having the following disadvantages:
(a) SEQ approach comprising regeneration/washing phases between the analyte contacting steps;
(b) constant analyte starting concentration c₀(A), which under some circumstances, when the inhibitor starting concentration c₀(I) is high, may lead to an almost complete inhibition of A (and actually does so!) and consequentially leads to the generation of a c_{eq}(A) which is practically not reliably measurable any longer; it has to be noted that also the reverse case, a constant inhibitor starting concentration c₀(I), can in principle generate a similarly negative effect;
(c) as a measure of c_{eq}(A) in the inhibition reaction solutions, the fitted association exponent of the binding of A to L (which is admittedly not generally valid) is used; it has to be noted that this exponent is indeed and definitely not directly proportional to c_{eq}(A).

The German patent application DE 100 05 301 A1 discloses MSK and requires a known analyte starting concentration c₀(A) and the ability to add, among others, competitors or inhibitors to the analyte. However, this is not done for determining the equilibrium constant of a preceding reaction. There exists also the possibility of using the evaluation of the curves to determine unknown analyte concentrations of samples, which is likewise executed not in order to determine the equilibrium constant of a preceding reaction. The contents of this patent application shall also be considered as part of the description.

The European patent EP 1 259 810 B1 discloses MSK and the determination of the unknown analyte starting concentration c₀(A) or c_{0,i}(A) one or multiple times. This is, however, not done for determining the equilibrium constant of a preceding reaction. It is simply a determination of concentrations of unknown samples. The content of this patent shall also be considered as part of the description.

The applications WO 2004/109284 A1 and U.S. 2005/0014179 A1 and U.S. 2005/0019933 A1 claim a MSK-like approach, which is meanwhile in practice known as Single-Cycle Kinetics (SCK). However, neither a reference to a determination of concentrations of unknown samples is given therein, nor is a reference made to the determination of the equilibrium constant of a preceding reaction.

A software program for evaluating biosensor data having been recorded under MSK conditions exists in the form of a prototype since summer 2005 and is meanwhile commercially (and also as a free trial) available from Kinomics GmbH (www.kinomics.com) for users of Biacore instruments. The program is virtually unlimited regarding the number, duration and concentration of the analyte contact steps and evaluates the biosensor data of each analyte contact step in detail (e.g. in respect to non-linearly fitted initial rates). Thus, the program is, after having received a comparative standard dilution series, usable for a comparative (i.e., a relative, but not an absolute-quantitative) evaluation of the analyte concentration of a corresponding dilution series of an unknown sample, but is not usable for determining the equilibrium constant of a preceding reaction.

The software of GE Healthcare for the MSK-like SCK approach meanwhile exists in two versions. A first, very limited version for old devices delivers, when performing data analysis, merely a general result, but does not deliver any separate, individual results for the successive analyte contact steps. For said reasons, a concentration determination for the individual analyte contact steps and a farther-reaching evaluation in respect to determining the equilibrium constant of a preceding reaction are not possible (and are not scheduled for implementation). A newer, less limited SCK software version for more recent devices does also not include an appropriate evaluation option.

### GENERAL DESCRIPTION

It is the object of the invention to provide for an easily handable method for determining the equilibrium constant of a preceding reaction by means of MSK. The term “preceding” in this context means that the MSK analysis is used to determine the equilibrium constant of a preceding equilibrium reaction, not that of an equilibrium reaction the MSK analysis itself is based on. In the following, said “preceding” equilibrium reaction is also referred to as “first equilibrium reaction” while the equilibrium reaction the MSK analysis is based on is referred to as “second” equilibrium reaction. The equilibrium constant being characteristic for the preceding or first equilibrium reaction is in the following referred to as the “first equilibrium constant” while the equilibrium constant of the succeeding MSK equilibrium reaction or “second equilibrium reaction” is in the following also referred to as “second equilibrium constant”. The present invention therefore represents an indirect approach for determining the first equilibrium constant by executing a MSK analysis of another equilibrium reaction. The term “equilibrium reaction” as used in the following encompasses any reversible reaction, the term does not imply that a measurement of said reaction has to be performed when said reaction is in equilibrium state.

The above object is achieved by a method comprising the features of claim 1.

A method for determining equilibrium constants in solution by Multi-Step Kinetics (MSK), which temporally preceded the subsequent analysis of the reaction, the method being characterized by the following steps:
that multiple different starting substances are brought in contact with each other in a preceding equilibrium reaction;
that the concentrations of mixtures of the different starting substances are chosen freely;
that solutions for the determination of the equilibrium reaction are then, in no particular order, by means of Multi-Step Kinetics and without any regeneration steps between the solutions of the measured equilibrium reactions, analyzed thereby for their respective initial rate or other measured values;
that the free equilibrium concentration of one or multiple starting substances is determined via their respective initial rate or via other measured values, and
that the equilibrium constant is determined via the LMA from the free equilibrium concentration and from the starting concentrations of one or more starting substances.

The previous paragraph means that multiple different types of starting substances, e.g. a first starting substance A and a second starting substance I are brought in contact with each other in a preceding, first equilibrium reaction; that the concentrations of mixtures of the different starting substances are freely chosen means that the method to which the invention relates comprises several embodiments according to which the concentrations of the starting substances (e.g. A and I) may differ relative to each other and may differ also with respect to their absolute starting concentrations.

The determination of initial rates by means of Multi-Step Kinetics without any regeneration steps between the individual solutions of the measured equilibrium reactions implies, that in the course of MSK analysis (of the second equilibrium reaction), several solutions are brought in contact with a ligand L, whereby at least one of the reactants, e.g. A, binds to the ligand L during the second equilibrium reaction being analyzed by MSK. The application order of the solutions to be analyzed via MSK can thereby be chosen arbitrarily.

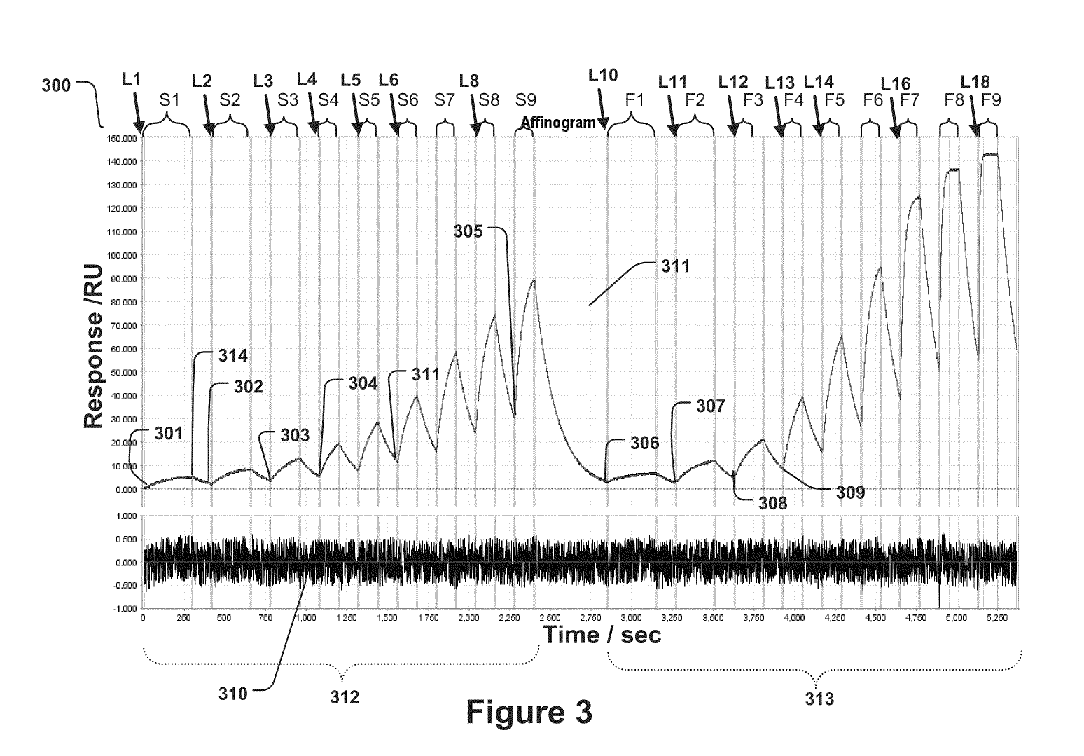

The concept of the initial rate is described in detail in the patent EP1259810 B1 and will be described in general terms with the help of the affinogram being exemplarily shown in FIG. 3.

An initial rate is the slope of the reaction signal curve at its basis point (or baseline). A basis point is a measuring time at which a sensor system that uses a ligand L has not yet been brought in contact with the binding partner of L, i.e., the analyte, so that at the basis point no complexes LA exist.

The slope can be determined e.g. by creating a tangent or a linear regression line in the basis point. The MSK method makes it possible to determine, by extrapolating the reaction signal curve to the basis point or baseline, the initial reaction rate of each respective reaction signal curve, even at a moment when the ligand partially has still bound some analyte, i.e., is not in a regenerated state.

The initial rate of a reaction signal curve at its origin (i.e., at the basis point or at the baseline) is obtained mathematically from the product of the exponent Kₒₙ of the measured reaction signal curve and its reaction signal R_{eq} in the final equilibrium state (i.e., in the equilibrium of the reaction).

The final value R_{eq} of the reaction signal R (R is plotted in FIG. 3 on the ordinate), which is asymptotically approached by the reaction signal curve, is called the final equilibrium state (synonymous names are equilibrium reaction signal and equilibrium response). R_{eq} does not necessarily have to be measured or recorded, it may also be determined based on the shape of the reaction signal curve. The reaction signal curve is exponential, as is well known in the art. The time course of the reaction signal R over time t (Rₜ) is given as:

Rₜ=R_{eq}×(1−e^{-k}^{ₒₙ}^{×t})

This reaction signal Rₜ progresses exponentially, beginning with a particular slope (the initial rate) at the basis point or the baseline and by asymptotically, with steadily decreasing slope, approaching the final equilibrium state R_{eq}; the mathematical equation of this nonlinear, exponential progress of the measured reaction signal curve is, according to the state of the art:

Rₜ=R_{eq}×(1−e^{-k}^{ₒₙ}^{×t})

with Rₜ=reaction signal (response) at time t, kₒₙ=exponent of the reaction signal curve, and t=time. In another aspect, this equation mathematically expresses that the slope of the reaction signal curve at time t=0 (i.e., the initial rate at the basis point) is equal to the product of R_{eq} and kₒₙ. After having recorded and stored the reaction signal curve, said curve (as well as any of the subsequent reaction signal curves) can be non-linearly adjusted (“approximated” or “fitted”) to the above mathematical equation by means of a computer program, thus making it possible to determine the two curve parameters R_{eq} and kₒₙ, which fully characterize the curve. This adjustment is also referred to as “non-linear fitting”. R_{eq} does not have to be reached for said purpose when performing the measurement, but can rather be forward-extrapolated via the fitting based on the partially recorded curve. Based on R_{eq} and kₒₙ, the non-linearly fitted initial rate of the curve (at the baseline) can be determined. This initial rate is, according to the state of the art, directly proportional to the analyte concentration.

MSK allows determining a multitude of parameters of the equilibrium reaction being analyzed by MSK, i.e., the second equilibrium reaction A+L⇄AL. Said parameters comprise, for example, the initial rate and the concentration of one or more starting substances A of said second equilibrium reaction at the moment of measurement, the concentrations having been determined via these initial rates. The MSK analysis method is described in detail in the patent EP 1259810 B1 whose content, as explained above, is part of this patent application, the individual steps of the MSK method and the determination of the initial rates are also described therein in detail.

An important advantage of the MSK method is, that MSK-measurements can be performed after a replacement of the analyzed solutions already before the equilibrium concentrations of ligand L and analyte A have been reached, which means that MSK is a particularly advantageous and fast method for determining the free concentration of the analyte A (ligand-binding partner) right at the moment of measurement.

By means of said initial rates or other measured values having been determined by the MSK analysis, the concentration of free (not being bound to L or to any other complexation partner such as I) A as present right at the moment of measurement can be determined. This determined free concentration of the analyte A, with A equally acting as starting substance (educt) of the first equilibrium reaction whose equilibrium constant is to be determined, is of great significance, because the determined free concentration of A at the moment of measurement has to be considered as the equilibrium concentration c_{eq}(A) with respect to the first equilibrium reaction. The fact that A is involved both in the second equilibrium reaction being analyzable by means of MSK and in the first equilibrium reaction makes it possible, via a MSK analysis based on the second equilibrium reaction, to make conclusions on the equilibrium constant of the first equilibrium reaction.

The L competes with the one or more other starting substances I of the first equilibrium reaction for binding to A. This means that the free concentration of the starting substance A measured by MSK is also equal to the equilibrium concentration c_{eq}(A) of this starting substance with regard to the first equilibrium reaction. This makes it possible in a further step to use the free concentration of A, having been determined by means of MSK, for determining the equilibrium constant of the first reaction by applying the LMA. The application of the LMA is possible because the starting substances and products of the first equilibrium reaction are present in their respective equilibrium concentration c_{eq}(A), c_{eq}(I), c_{eq}(AI).

The number of the different starting substances in the preceding equilibrium reaction thereby is two. The mixtures of the different starting substances consist of mixtures of analyte and inhibitor/competitor.

The other measured values are the exponent or the final equilibrium state.

The exponent is the exponent kₒₙ as described in EP 1259810 B1.

The final equilibrium state R_{eq} is the measured equilibrium value R′_{eq} described in EP 1259810 B1, which indicates how much analyte has ultimately bound to the ligand in the second equilibrium reaction. The amount of bound analyte can thereby be expressed e.g. in units of RU (resonance unit) when the binding of A to L is achieved e.g. by means of biosensors. 1 RU corresponds to approximately 1 pg Analyt/mm² sensor surface in the event that the analyte is a protein. Other units, for example g/mm², are also possible, depending on the used ligand-binding assay or measuring system. Besides using biosensors, RIA or ELISA assays can likewise be used, whereby in these cases R_{eq} is detected via appropriate methods of measurement (e.g. autoradiograph, scintillation counter, fluorescence microscope, etc.). All these methods make it possible, based on the amount of bound analyte A having been determined by MSK, to deduce the amount of analyte in solution and thus its concentration and to use these measured values to determine the first equilibrium constant. Thereby, it is of essential importance that when using MSK it is not necessary to wait until the final equilibrium state R_{eq} is reached, but rather it is possible to extrapolate it from the measured reaction progress over time. In principle, the solutions can be measured in any order; an increasing order however facilitates the succeeding analysis. Therefore, in order to determine the equilibrium reaction, the solutions are measured preferentially in the order of increasing starting concentration of one of their starting substances by means of the Multi-Step Kinetics and without regeneration steps between the individual solutions. A reference measurement is provided, which is used for comparison purposes.

A conversion of the starting concentrations in real equilibrium concentrations based on the use of the equilibrium constant is performed by means of a quadratic equation.

In the following, embodiments shall elucidate the invention and its advantages in a general manner and then specifically with reference to the enclosed figures. Particular emphasis is put on the advantage of using identical starting concentrations of the starting substances respectively and, respectively, on the resulting benefits (in principle, however, these starting concentrations can be any arbitrary concentration, provided the described complete analyte inhibition being avoided; this means in particular that keeping one starting concentration constant in relation to that of another should be considered with great caution). The embodiments are also intended to illustrate once again that the method presented herein is not about determining the kinetics or thermodynamics of the reactions between A and L on the sensor (as is the case in conventional MSK), but rather is about determining the equilibrium constant of the preceding reaction between A and I (whereby MSK is just a means to an end).

Starting from a series of preceding, e.g., inhibition, reactions with preferably the same respective starting concentrations c₀(A) and c₀(I) and, if necessary, followed by non-inhibited analyte solutions as an internal reference standard, an inhibition in solution assay based on the MSK principle is performed against L on the sensor surface, after which the actual free equilibrium concentration of A, c_{eq}(A), and/or of I, c_{eq}(I), in solution is determined based on a non-linear fitting of the initial rates, and based on this result via the LMA the equilibrium constant of the preceding reaction is determined (the equilibrium concentration of the reversibly formed analyte-inhibitor complex, c_{eq}(AI), follows here from the difference between the starting concentration and equilibrium concentration of A or I). According to the LMA, it is:

$${K}_{1} = \frac{{c}_{eq} ( A ) \times {c}_{eq} ( I )}{{c}_{eq} ( AI )} = constant$$

(i.e., K must in theory be identical for all preceding reaction solutions irrespective of c₀(A) and c₀(I)).

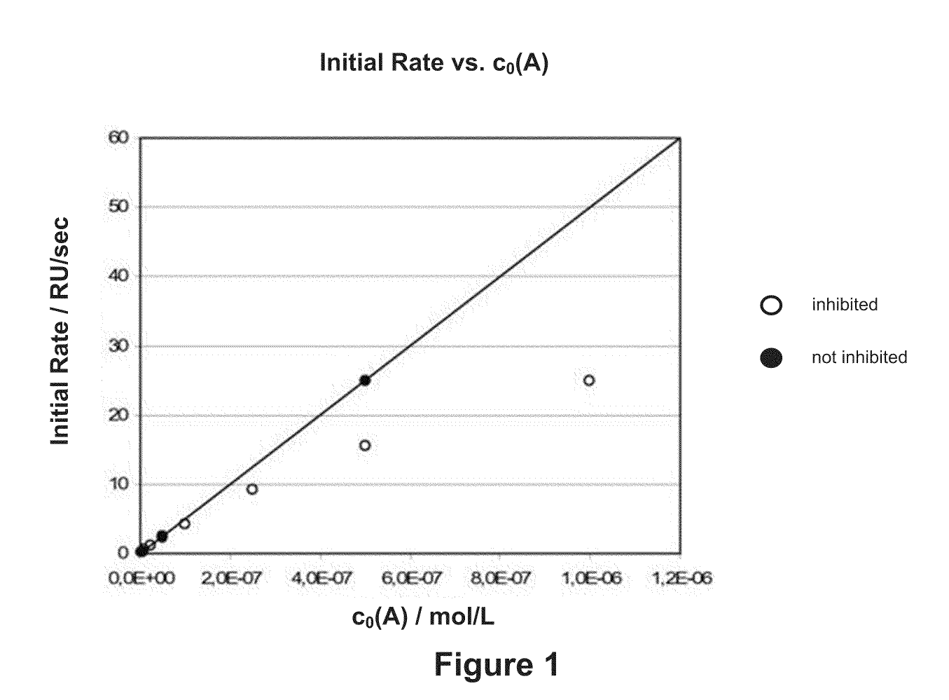

As for said reason all equilibrium concentrations of the involved reactants are known (i.e., can be determined), for each of the inhibition reactions the equilibrium constant, and based upon these values, their reliable, arithmetic mean, including the standard deviation, can be determined. This is shown below in detail for a preferred embodiment by making reference to FIG. 1 and the next paragraphs.

In one further aspect, whereby the invention relates to a method for determining a first equilibrium constant of a first equilibrium reaction, the method comprises the following steps:
Providing two or more solutions, each of these solutions comprising all first educts and first products involved in the first equilibrium reaction, the first educts and the first products of the first equilibrium reaction being referred to as first reactants, whereby the first reactants of each respective solution are in reaction equilibrium of the first equilibrium reaction, whereby in the reaction equilibrium of the first equilibrium reaction the first reactants are in their respective equilibrium concentration;
Providing a ligand, which together with at least one of the first reactants, which is called analyte, can be involved in a second equilibrium reaction, whereby the ligand and the analyte each act as second educts of the second equilibrium reaction;
Bringing together the two or more solutions and the ligand in thus a way that the second equilibrium reaction takes place;
Conducting a MSK analysis of the second equilibrium reaction taking place with the two or more solutions, whereby a MSK result is determined;
Determining the first equilibrium constant by using the result of the MSK analysis of the second equilibrium reaction.

According to embodiments, the first equilibrium reaction is, for example, an association reaction A+I⇄AI or a dissociation reaction AI⇄A+I. For the sake of simplicity, the educts and products of the below-mentioned reaction equations have the stoichiometric factor 1.

However, the present invention also covers more complex reaction equations comprising more than two educts and/or products and stoichiometric factors >1, the concentrations to be calculated in these cases can be mathematically derived accordingly via the LMA.

According to embodiments of the invention, the number of the first educts is two and the analyte is an educt of the first reaction equation.

According to embodiments of the invention one of the two first educts is the analyte and the other of the two first educts is an inhibitor.

The term inhibitor is to be understood in the following as to include also competitors. An inhibitor is a substance that prevents the analyte A from (covalently or non-covalently) binding to ligand L. The inhibitor thereby exerts its effect by interacting with the analyte at the same or at a different binding site as used by the ligand, while a competitor interacts with the same binding site of the analyte as the ligand and thereby competes with the ligand for forming a complex with A.

According to embodiments of the invention, the MSK result is determined or calculated based on a MSK exponent kₒₙ, based on a final equilibrium state R_{eq}, based on a plot comprising measured initial rates for different starting concentrations of the analyte or based on an initial rate at a basis point. The basis point is indicative of a moment in a MSK analysis when the MSK analysis is started and when the ligand has essentially not bound any analyte yet. A line having been horizontally plotted at the basis point (parallel to the x-axis) is called baseline. A turning point is indicative of a moment during a MSK analysis when a new solution comprising a higher or lower analyte concentration than the previously analyzed solution is brought together with the ligand, whereby no regeneration step for regenerating the ligand is performed or is necessary in the meantime between applying the solutions. The method according to these embodiments further includes the following steps:
Determining the equilibrium concentration c_{eq}(A) of the analyte based on the MSK result, and
Calculating the first equilibrium constant from the equilibrium concentration c_{eq}(A) of the analyte by applying the law of mass action.

According to preferred embodiments, the two or more solutions are brought together with the ligand and analyzed in the MSK analysis in the order of increasing starting concentration of the analyte.

According to embodiments of the invention, the MSK result is used together with a reference line to calculate or to graphically determine the first equilibrium constant. The reference line describes initial rates (having been determined e.g. in a further MSK analysis) in dependence on the starting concentration c₀(A) of the analyte, whereby in respect to said dependence described by the reference line only the second equilibrium reaction, not the first equilibrium reaction, is considered. This means that for determining the reference line the first equilibrium reaction is not considered.

According to embodiments of the invention the reference line is created by performing one of the following steps or procedures:
Reading the reference line having been stored on a data storage medium;
Reading reference values having been stored on a data storage medium in order to automatically calculate a regression line for the read reference values, whereby the calculated regression line is used as a reference line; or
Performing a reference measurement with multiple additional solutions, whereby the additional solutions comprise the second reactants of the second equilibrium reaction, but apart from the analyte do not comprise reactants of the second equilibrium reaction, whereby the solutions are brought together with the ligand and whereby the initial rates are determined for multiple starting concentrations of the at least one first educt, and whereby a regression line for the recorded reference values is determined.

According to embodiments, the reference measurement is performed in the form of a MSK analysis.

According to embodiments of the invention, the starting concentrations of the two first educts are identical in each of the two or more solutions.

According to embodiments of the invention the ligand is immobilized. Depending on the embodiment, the MSK analysis is executed based on using one of the following assay approaches: ELISA, RIA or biosensors.

In another aspect, the invention relates to a method for determining and visualizing the strength of a first inhibitor, whereby the first inhibitor inhibits, by binding to an analyte, the binding of the analyte to a ligand, the method comprising the following steps:
Displaying a plot whose ordinate is indicative of measured initial rates of linearly increasing value, and whose abscissa is indicative of starting concentrations c₀(A) of the analyte of linearly increasing value,
Drawing a reference line in the plot, whereby the reference line indicates the initial rates which exist at given starting concentrations c₀(A) of the analyte;
Drawing two or more first values on the plot which were determined in a first MSK analysis of two or more first solutions, whereby in these two or more first solutions the analyte and the first inhibitor were dissolved, whereby each first value is indicative of an initial rate for a given starting concentration c₀(A) of the first analyte in one of the two or more first solutions,
Determining a point P₁₅₀ in the plot,
   whereby P₁₅₀ is one of the two or more first values or is a value on a regression curve through the two or more first values,
   whereby P₁₅₀ is determined as a point P₁₅₀ whose ordinate value is essentially half as high as the ordinate value of the reference line at a starting concentration c₀(A) of the first analyte;
Determining the inhibitory strength of the first inhibitor as a result value, which is inversely proportional to the starting concentration values c₀(A) of the first analyte at point P₁₅₀, thus the P₁₅₀ of a strong inhibitor is displayed in the plot further to the left compared to a P₁₅₀ of a weak inhibitor.

According to further embodiments, the strength of the first inhibitor can clearly be compared visually with the strength of a second inhibitor, whereby the visualization method further comprises the following steps:
Drawing additional two or more second values which were determined in a MSK analysis of two or more second solutions, whereby in these two or more second solutions the analyte and a second inhibitor were dissolved, whereby each additional second value is indicative of an initial rate for a given starting concentration c₀(A) of the analyte in one of the two or more second solutions,
Determining a first regression curve for the two or more first values,
Determining a second regression curve for the two or more second values.
Determining that one inhibitor as being the stronger inhibitor, which at any given starting concentration c₀(A) of the analyte has a lower initial rate than the other inhibitor. The latter means that the stronger inhibitor has a lower ordinate value for any arbitrary abscissa value (except for the origin).

According to embodiments of the invention, the plot is displayed on a graphical user interface of a computer system, e.g. a screen, or is displayed by means of a printout on paper or another medium. The computer system comprises a graphical user interface, e.g. a screen and/or a printer, a processor and a computer-readable, non-volatile storage medium, e.g. an electromagnetic memory or a flash drive. The computer readable storage medium comprises instructions that, when executed by the processor, result in the visualization of the inhibitor strength according to one of the described embodiments.

According to embodiments of the invention, the two or more first values that are used for visualization are determined by executing the following steps:
Providing the two or more first solutions, each first solution comprising the first inhibitor and the analyte, whereby the first inhibitor and the analyte are involved as first educts in a first equilibrium reaction, whereby at the reaction equilibrium of the first equilibrium reaction the first educts and the first products of this first equilibrium reaction are in their respective equilibrium concentration, whereby the concentration of the analyte is different in each of the two or more first solutions;
Providing a ligand, which together with the at least one analyte can be involved in a second equilibrium reaction, the ligand and the at least one analyte each acting as a second educt of the second equilibrium reaction;
Bringing together the two or more first solutions and the ligand, so that the second equilibrium reaction takes place;
Conducting a first MSK analysis of the second equilibrium reaction taking place in the two or more first solutions for determining two or more initial rates for the two or more first solutions, the initial rates being returned as first values;

The determination of the second values for the second inhibitor is performed analogously.

FIG. 1 shows exemplary the calculated initial rates of non-inhibited analyte solutions, which are represented by dots and which constitute the corresponding reference. The initial rates of inhibited analyte solutions, which are represented by circles, are plotted against the analyte starting concentration c₀(A), whereby a 1:1 inhibition of A and I with equally high starting concentrations c₀(A) and c₀(I) respectively is used. The equilibrium concentrations c_{eq}(A) resulting from the initial rates of the inhibited analyte solutions via the reference provide, via the LMA, for an average value of K_{I}=5×10⁻⁷ mol/L for the preceding equilibrium. Overall, this method is (in addition to the abandonment of the unnecessary SEQ-regeneration/washing phases) much easier, because:

Starting from a series of preceding inhibition reactions with respectively equal starting concentrations c₀(A) and c₀(I), and being followed by non-inhibited analyte solutions as internal reference standard, an inhibition in solution assay based on the MSK-principle is performed against L on the sensor surface. Then, by a non-linear fitting of the initial rates of the inhibited solutions (and in relation to the initial rates of the internal reference standard used as a reference), their respective actual (free) equilibrium concentration of A, c_{eq}(A), and thus that of I, c_{eq}(I), in solution are determined, and based thereupon, the equilibrium constant of the preceding reaction is determined via the LMA. The equilibrium concentration of the reversibly formed analyte-inhibitor complex, c_{eq}(AI), is, as was explained above, obtained as the result of the difference between the starting concentration and the equilibrium concentration of A or I.

Using preferentially the same respective starting concentrations c₀(A) and c₀(I) and measuring them, their measurement being succeeded by non-inhibited analyte solutions, has several advantages which will here be explained using the example of a 1:1 inhibition between A and I.

As outlined above, if c₀(A) or, instead, c₀(I), is kept constant while the respective other starting concentration is varied, such a strong inhibition may happen, that the remaining extremely low c_{eq}(A) yields a measurement result which gets virtually lost in the noise of the instruments. In case of equal c₀(A) and c₀(I), this cannot happen as a matter of principle, meaning that a low c₀(A) is damped only slightly, thus appearing as almost non-inhibited. Even a higher c₀(A) is usually damped only about 50 to 80% and is therefore still reliably measurable.

Depending on the inhibition between A and I, which needs to be estimated in a preceding experiment if required, the concentrations of the non-inhibited analyte-reference solutions can be chosen such that their measurement results (being of advantage for the following evaluation) still lie safely above those of the inhibited higher c₀(A).

The final measuring of not-inhibited analyte reference solutions in the same MSK analysis is not strictly necessary, but has the advantages that, on the one hand, if L needs to be prepared on the sensor surface for each analysis anew, different occupation densities of L on the sensor surface are compensated and that, on the other hand, for the sake of practical considerations regarding the clarity of presentation, inhibited and non-inhibited analyte solutions can be directly compared with each other in a graph.

The MSK software of Kinomics GmbH makes it possible, for example, to graphically plot the initial rates of all measured analyte solutions against their starting concentration c₀(A). Here, one can selectively linearly fit e.g. only the rates of the non-inhibited analyte reference solutions. Ideally, this should yield a straight line, as the initial rate is directly proportional to the actual analyte concentration and thus is a valid measure thereof. All rates of inhibition reactions—having also been computed and displayed, but not having been fitted accordingly—will now (because they are plotted against their c₀(A) value, but are damped because of their decreased c_{eq}(A) value after inhibition) lie below the reference line.

This advantage mentioned in the above paragraph is in practical terms one of the most useful of the present invention. By looking at the plot shown in FIG. 1 it is easily recognizable in a quantitative manner where the equilibrium constant lies. That inhibited starting concentration c₀(A), here

$$1 \times {10}^{- 6} \frac{mol}{L} ,$$

yielding precisely half the initial rate compared to the non-inhibited reference line (25 instead of 50 RU s⁻¹), represents with the actual analyte concentration

$${c}_{eq } ( A ) = 5 \times {10}^{- 7} \frac{mol}{L}$$

corresponding to its rate, according to the LMA, exactly the

$${K}_{1} = 5 \times {10}^{- 7} \frac{mol}{L} .$$

In other words, already by a mere visual inspection of the plot, the degree of inhibition can be determined—“strong” inhibitors lie with a 50% reduction of the initial rate (i.e., 50% inhibition of the analyte concentration corresponding to its K_{I} value) to the left of the plot, the “weak” inhibitors lie, however, to the right.

Based on the internal reference line, as can be seen in the depictured plot of FIG. 1, it is possible to infer via its slope from the measured initial rate of inhibited analyte solutions their concentration. According to the above-mentioned LMA relationship, it is possible to determine the equilibrium constant of a preceding reaction of any kind, such as e.g. the described 1:1 inhibition.

From the non-linearly fitted initial rates it is possible, as shown above, in a simple manner, to determine the actual free equilibrium concentration of A, c_{eq}(A), and ultimately the K_{I}-value—whether manually, by means of a simple (e.g. Excel) macro or by means of a corresponding extension to the existing MSK software.

In addition, the above mentioned mean value of the K_{I}-value can also, via a quadratic equation, and also automatically, be used to correct the initial c₀(A)-values having been plotted in the above mentioned plot into the actual c_{eq}(A)-values. The measuring value scattering will thereby be preserved. Ideally, the initial rates of the inhibited and non-inhibited analyte solutions should, regard being had to their measuring value scattering, reside on a straight line.

The preferably equally high starting concentrations c₀(A) and c₀(I) can be chosen arbitrarily different from each other provided that (as discussed) a complete analyte inhibition is avoided. A special case arises if, in order to further evaluate inhibited analyte solutions, the final equilibrium states of said solutions of in the MSK analysis are used (for example, by considering the corresponding final states of non-inhibited analyte solutions as a reference). For a known K_{D} value of the equilibrium between L and A, further preferred starting concentrations are: c₀(A) remaining constant=K_{D} while c₀(I) is varying, as in this case a non-inhibited c₀(A) in the MSK analysis returns as final equilibrium state precisely the half-maximal saturation of L on the sensor surface. Thereby, it is important to keep the c₀(I) range narrow or low enough to exclude a complete inhibition of the analyte.

The components of the preceding equilibrium reaction can be chosen arbitrarily, such as A and I, A and several different I, A and L, possibly under addition of further substances which can influence the reaction in the preceding equilibrium and/or can influence the reaction during the MSK analysis. The ratio of reacting starting substances of the preceding equilibrium is thereby not limited to a ratio of 1:1, but can be arbitrary unequal. The MSK analysis is not limited to the detection of A, but may at the same time or alternatively focus on one or more components of the preceding equilibrium.

Determining the equilibrium constant of a preceding reaction via a MSK analysis is not limited to the evaluation of the initial rates of the analyte solutions, but can also be based on the evaluation of other measuring values (e.g. exponents or final equilibrium states or combinations of measuring values), which are obtained when fitting the formation rate curves (these are the individual association and dissociation curves in MSK).

In addition, the MSK-software of Kinomics GmbH already today quantitatively detects such analyte contact steps, which, for example due to so-called mass transport limitation (MTL), run in a damped manner per se (because, for example, the analyte is bound by L on the sensor surface faster than it is replenished by the biosensor pumping mechanism), resulting in measurement data no longer behaving according to the required linearity. Suchlike device-specific artifacts can, due to their detectability by the MSK-Software, be identified by the MSK-Software and specific measurement parameters can be adjusted accordingly, or specific concentration ranges of A and/or I can be excluded from the analysis.

In principle, the raw data (e.g. initial rate vs. c₀(A) or vs. c_{eq}(A) or other graphical representations of a measuring value) may furthermore be directly evaluated by corresponding pre-defined algorithms in respect to the equilibrium constant to be determined.

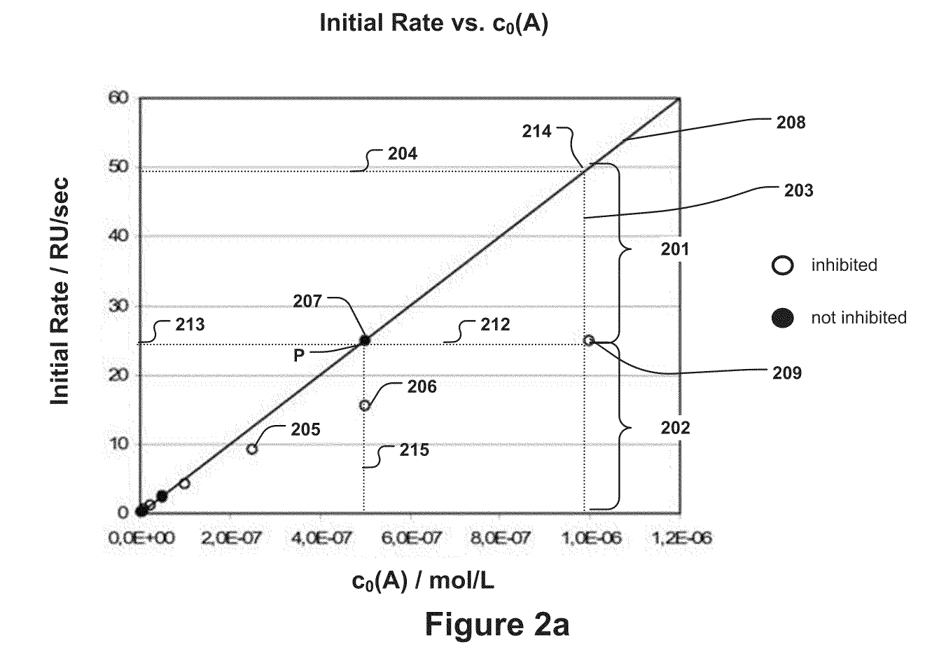

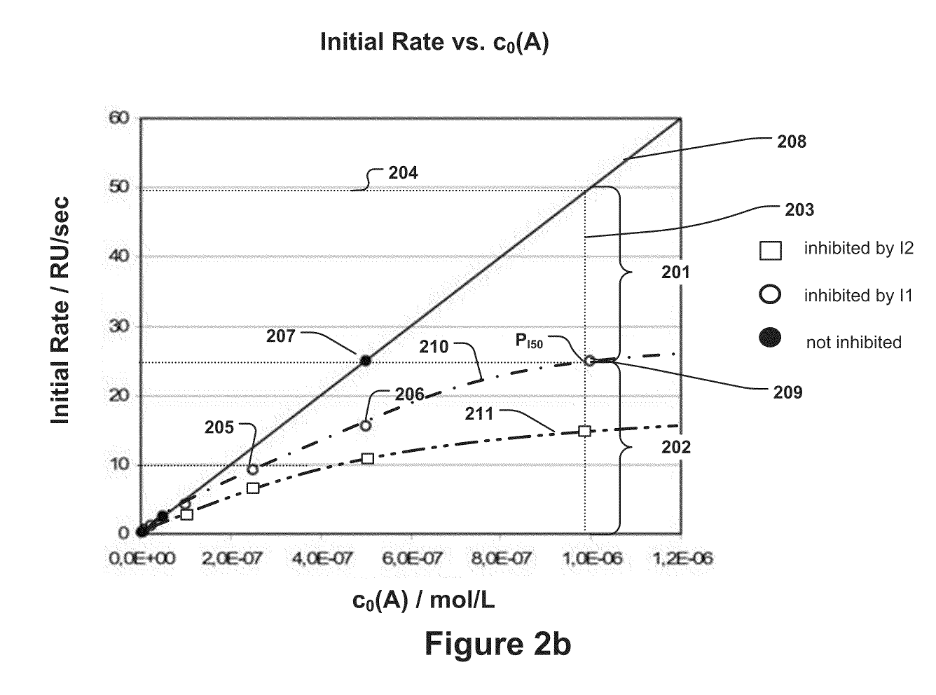

FIG. 2a shows a plot according to FIG. 1, whereby additional reference symbols and lines 204, 212 are supposed to assist in explaining the determination of ordinate intercepts 201, 202.

By means of the displayed plot the strength of an inhibitor I can be recognized quickly and intuitively. It is even possible to read the equilibrium constant K_{I} of the first equilibrium concentration directly from the abscissa provided the starting concentrations of A and I were equal in the respective solutions.

The ordinate of the plot thereby indicates the height of the initial rate (in this example e.g. in the unit RU). The abscissa indicates the starting concentration c₀(A) of the analyte, which can bind both to the ligand according to a second equilibrium reaction and to the inhibitor according to a first equilibrium reaction.

In a first MSK analysis, for each of a multitude of first solutions containing the analyte A and the inhibitor I in their respective equilibrium concentration, the initial rate is determined and plotted in the plot. The thus obtained measuring values are plotted as circles and are referred to as ‘inhibited measuring values’, because the total amount of A was not available for binding to L, since at least a part of the A was bound to the I and thus existed in an “inhibited” form.

In addition, a reference line 208 is plotted in the plot. The reference line refers to the initial rate which would have been expected for each given starting concentration c₀(A), if the solution used for the MSK measurement would not have contained any inhibitor, hence if the analyte A had existed in an non-inhibited form. Depending on the embodiment, this reference line may have been determined for a particular analyte A beforehand, as described for example for FIG. 1, and may for example have been saved in a computer program.

According to further embodiments, the reference line is created by the following steps which may be carried out before or after the first MSK analysis:

Performing a second MSK analysis with several additional solutions that contain solely the analyte A and not the inhibitor I, whereby in each case the initial rate is determined and the determined first measuring values are plotted in the plot.

In the embodiment shown in FIG. 2a, the thus determined measuring values are depicted as dots (not inhibited). By manual or computer-aided determination and plotting of a linear regression line between the measuring values having been obtained in the second MSK analysis, the reference line 208 is determined. Storing the reference values or the reference line for use in a MSK analysis to be carried out at a later stage, however, requires that the ligand or a surface on which the ligand is immobilized, during the execution of the MSK analysis for determining the reference line does not significantly differ from the ligand or said surface which is used for the (indirect) determination of the first equilibrium constant, which means that neither less nor more ligand is present and that the percentage of the ligand being available for binding A is not significantly different from said percentage of the ligand at the time of measuring the reference values.

Executing the second MSK analysis for determining the reference line can be done before or after the first MSK analysis. Preferably, it is executed afterwards, because the free concentration of A in the non-inhibited solutions for the second MSK is greater than the free concentration of A in the solutions of the first MSK, which is metrologically advantageous because such an approach allows bringing ligand L in contact with solutions of increasing analyte concentration in a stepwise fashion.

According to the embodiment shown in FIG. 2a, which is particularly advantageous in terms of its visualization, the starting concentrations c₀(A) and c₀(I) are respectively equal in each of the solutions used in the first MSK, but the c₀(A) and c₀(I) of the various solutions differ from each other. Preferably, the solutions are analyzed in the order of increasing c₀(A) (and thus also of increasing c₀(I)) and the measured values are plotted in the plot.

The measuring point 209 is a particular measuring point, as it divides the ordinate intercept between the abscissa and the abscissa point 214 in two equal parts, a first intercept 210 and a second intercept 202. At that analyte concentration c₀(A) being represented by point 209 the initial rate is reduced by the examined inhibitor by 50% compared to the non-inhibited solution. This in turn implies that at this analyte concentration half of the analyte exists in the form of an AI complex (while the non-inhibited point 214 on the reference line corresponds to an initial rate of 50 RU, the measuring point 209 corresponds to a half initial rate of 25 RU (ordinate value 213). On the basis of this property, the point 209 is also referred to as point P₁₅₀.

By plotting a ledger line 215 between the intersection of reference line 208 and the auxiliary line 212 (which represents the ordinate value of the measuring point 209) it is possible to read the actual analyte concentration

$${c}_{eq} ( A ) = 5 \times {10}^{- 7} \frac{mol}{L}$$

directly from the abscissa. Since at the time of measurement the analyte was in equilibrium with the inhibitor, i.e., existed according to its equilibrium concentration for the first equilibrium reaction (A+I⇄AI), according to the LMA the value of

$$5 \times {10}^{- 7} \frac{mol}{L}$$

being read from the abscissa exactly corresponds to the k of the first equilibrium reaction:

$${K}_{1} = 5 \times {10}^{- 7} \frac{mol}{L} .$$

The plot thus allows, identical starting concentrations c₀(A) and c₀(I) provided, to directly read the K_{I} value as the point of intersection of the ledger line 215 with the abscissa, whereby the ledger line is a vertical line that intersects with the reference line at a point P, whereby the ordinate value of the point P is equal to the ordinate value of that very point P₁₅₀ whose ordinate value is half the ordinate value of the reference line at the same abscissa value as said point P₁₅₀.

FIG. 2a makes it clear that the measuring points 205 or 206 do not divide the ordinate value of the reference line at the respective analyte concentration c₀(A) into two halves. A strong inhibitor is characterized in that the measuring point at which the first ordinate intercept 201 and the second ordinate intercept 202 are of the same size respectively is plotted already at low c₀(A), i.e., is plotted on the left side of the plot.

In FIG. 2b, two inhibitors I1 and I2 were studied and the corresponding measuring values were plotted. In the following, in the description of FIG. 2b the terms “first” and “second” MSK analysis will also be used, although the second MSK analysis is not used for determining the reference line, but rather for determining the initial rates for a second inhibitor (which means that these initial rates were obtained by MSK analysis of solutions containing the analyte A and the second inhibitor).

At first, a reference line 208 was determined by means of MSK experimentally or was read from a data storage medium and plotted. In addition, a first series of MSK measurements was performed for multiple solutions containing both A and I1 in identical concentrations in each respective solution. A second series of MSK measurements was carried out for multiple solutions containing both A and I2 in identical concentrations in each respective solution. The measuring values having been obtained for the first and second series of measurements were plotted separately for I1 (circle) and I2 (square) in the plot and a regression curve was determined.

It can be derived easily from FIG. 2b that the inhibitor I2 is stronger than the inhibitor I1, as the measuring values and/or the regression curve 211 corresponding to I2 lie below the measuring values and/or regression curve 210 of I1.

FIG. 3 shows the determination of an equilibrium constant K_{I} by means of a MSK analysis executed on a multitude of solutions, whereby the results are plotted on the affinogram 300. The affinogram comprises the reaction progress of an equilibrium reaction between an analyte A and a ligand L having been measured over time (abscissa), whereby the reaction progress is plotted in the form of a reaction signal (ordinate). The background noise signal 310 of the reaction signal detector is shown at the bottom of the plot. A reaction signal indicates the amount of analyte being bound by the ligand at the moment of measurement. The amount of bound analyte can be specified for example in grams per mm² sensor surface, in mol per mm² or in RU. In the embodiment shown in FIG. 3, a ligand having been immobilized on a biosensor was used for determining a reaction signal (here: a so-called “response” with the unit RU-“Resonance Units”). The immobilization can be accomplished for example by capturing methods, e.g. based on the streptavidin-biotin system.

In other embodiments, the MSK analysis is performed based on other ligand- or other detection systems. The ELISA approach, for example, comes into question, whereby determining the degree of loading the ligand with analyte, for example, may be done by fluorescence measurement. RIA based approaches can also be used, whereby the reaction signal can be detected, for example, by a scintillation counter. A reaction signal could in this case be specified, for example, in radioactive decays per time unit.

The entire process can be divided into two main passages 312 and 313, whereby preferentially the first passage 312 is performed prior to the second passage 313. However, the implementation of the second passage 313 prior to the first passage 312 is also possible and it is also possible to execute the two passages with a long time interval in between, provided the nature of the ligand or of a sensor surface coated with the ligand used in the two passages is largely the same, i.e., the number, density and complexation of the ligand are essentially the same. Furthermore, the second passage, if executed in a highly reproducible manner, may be executed and recorded only occasionally or once and can be stored for later analyses and comparisons.

The first passage 312 represents a MSK analysis for determining the equilibrium concentration of an analyte in a multitude of solutions L1-L9. The second passage 313 represents a MSK analysis for determining a reference line or a set of reference values.

The solutions L1-L9 each contain all of the reactants of a first equilibrium reaction A+I ⇄AI, whereby the first equilibrium reaction in each of the individual solutions L1 to L9 is in its reaction equilibrium and thus the reactants A, I, and AI are present in their respective equilibrium concentrations c_{eq}(A), c_{eq}(I) and c_{eq}(AI), which are unknown. The starting concentrations c₀(A) and c₀(I) of the respective solutions are known, however, e.g. from the manufacturing process of the solutions.

Preferably, the starting concentration of the analyte A and the inhibitor I in the respective solutions L1-L9 are equal, i.e., A and I are present in each particular solution in a 1:1 ratio. The concentrations of the analyte or inhibitor of the individual solutions are, however, different from each other. Preferably, the solutions L1 to L9, after the reactants of the first equilibrium reaction in each of the solutions being present in their respective equilibrium concentrations, are added in the order of increasing analyte concentration. In the embodiment shown in FIG. 3, given the solutions L1-L9, the solution L1 has the lowest analyte concentration, L9 the highest.

The time periods S1-S9, during which the free analyte A can bind to the ligand L (the reaction equilibrium of the second equilibrium reaction between A and L does not have to be reached) preferably are longer for small analyte concentrations. Accordingly, S1 and S2 are longer than the following time periods S3 to S9. An advantage of the MSK analysis approach is, that it is not necessary to implement a regeneration phase between the analysis of the individual solutions, i.e., a phase in which the analyte can completely dissociate from the ligand, thereby regenerating the ligand. Rather, it is possible, based on the course of the reaction signal curves, as explained below, to calculate that initial rate which would exist at a given concentration of the analyte, if the ligand would have been completely unoccupied at the moment of measurement (basis point).

After having applied a solution on the ligand, as depicted in FIG. 3 by the black arrows at the upper edge of the plot, the time periods S1-S9 succeed, in which the free analyte A of the individual solutions respectively interacts with the ligand, binds to the ligand and thus forms a complex LA. Throughout the whole MSK analysis in passages 312 and 313, the reaction signal is measured continuously and plotted over the time.

This reaction signal progresses in an exponential manner, beginning with a particular slope (the initial rate) at the basis point or the baseline and asymptotically, with steadily decreasing slope, approaching the final equilibrium state R_{eq}; the mathematical equation of this nonlinear, exponential course of the measured reaction signal curve is, according to the state of the art:

Rₜ=R_{eq}×(1−e^{-k}^{ₒₙ}^{×t})

with Rₜ=reaction signal (response) at time t, kₒₙ=exponent of the reaction signal curve, and t=time. This equation, on the other hand, mathematically indicates that the slope of the reaction signal curve at time t=0 (i.e., the initial rate at the basis point) is equal to the product of R_{eq} and kₒₙ. After having recorded and stored the reaction signal curve, the reaction signal curve (as all subsequent reaction signal curves) can be non-linearly adjusted (“approximated” or “fitted”) to the above mentioned mathematical equation by means of a computer program, thus returning the two curve parameters R_{eq} and kₒₙ as a result, which fully characterize the curve. This adjustment is also referred to as “non-linear fitting”. It is not necessary that R_{eq} was reached during the measurement, it rather can be extrapolated via the fitting from the partially recorded curve. From R_{eq} and kₒₙ, the non-linearly fitted initial rate of the curve (at the baseline) can be deduced. This initial rate is, according to the state of the art knowledge, directly proportional to the analyte concentration. This also applies to all the succeeding reaction signal curves, although they do not start at the baseline; the characteristics of a curve described by the two parameters R_{eq} and kₒₙ is constant, thus the curve (in correspondence to its forward-extrapolation to R_{eq}) can likewise be used for extrapolating backwards to its non-linearly fitted initial rate (at the baseline).

By measuring the initial rate of the reaction signal at the basis point 301 (t=0) and by extrapolation backwards from all turning points 302, 303, 304, . . . , 305, the initial reaction rate of the reaction signal curve is determined for each of the measured solutions. For determining the initial rate of the solution L3, the reaction signal curve would be back-extrapolated to the baseline starting from turning point 303.

At the baseline, the initial rate is proportional to the analyte concentration. This does not apply to the turning points 302, 303, 304, . . . , 305 Never the less, it is possible by means of MSK (as described in EP 1259810) to determine the initial rates for the reaction signal curves also for the solutions L2-L9, although the ligand at the time of adding said solutions at the turning points 302, 303, 304, . . . , 305 is not present in a fully regenerated form.

Thus, in the first passage 312 of the measurement approach, for each of the solutions L1-L9 an initial rate is determined for the starting concentration c₀(A) of each solution respectively analyzed, whereby the reaction signal (measured in this embodiment as a response) is measured by means of a MSK analysis. The determined initial rates are inhibited initial rates, since the inhibitor, which is contained in each of the solutions L1-L9, forms complexes with fractions of the free analyte A, so that ultimately less free analyte is available for binding L as would be the case when the solutions contained only the analyte and not also the inhibitor. The determined inhibited initial rates can be plotted in a further step in plots in accordance with FIGS. 1-2B (as circles).

Upon completion of the first passage 312 of the analysis, a somewhat extended intermediate step 311 follows, in which the ligand is brought in contact with a solution which does not comprise analyte A—as had already been the case between time points 314 and 302 and corresponding succeeding periods with declining reaction signal curves, which may be necessary for metrological reasons but which are not needed for the further evaluation procedure.

In the second passage 313 of the analysis, reference measuring values of initial rates are determined based on solutions L10-L18, whereby the reference measuring values may be used for creating a reference line. The solutions L10-L18 merely contain the analyte, but no inhibitor. Thus, the analyte A is present in its non-inhibited form. The solutions L10-L18 are brought in contact with the ligand in the order of increasing analyte concentration in these solutions and the course of the second equilibrium reaction is recorded. In phases F1-F9, the analyte can thereby bind to the ligand, whereby no inhibitor can prevent the binding of the analyte. The initial rates are also determined here, as described above, and can be plotted in a plot according to FIGS. 1-2b. The points 306-309 each represent turning points based on which the reaction signal curves are back-extrapolated to their respective initial rates at the baseline.

The equilibrium concentrations of A in solutions L1-L9 are not known, but they can be determined indirectly by determining reference values during the second passage 313 and by comparing the determined inhibited initial rates with these determined reference values. By this comparison, the equilibrium concentration of the analyte in the presence of a specific amount of an inhibitor can be calculated, which in turn allows drawing conclusions on the equilibrium constant of the equilibrium reaction between A, I and AI via the LMA. The invention was described by making reference to a preferred embodiment. However, it is conceivable to one skilled in the art that variations or modifications of the invention can be made without departing from the scope of the following claims.

1. A method for determining an equilibrium constant of a first equilibrium reaction, the first equilibrium reaction preceding a subsequent analysis of this reaction, characterized by the following steps:
that a plurality of starting substances mixed in multiple solutions are brought in contact with each other, whereby the starting substances in each of the respective solutions are at least a first starting substance I and a second starting substance A;
whereby the ratio of the starting concentrations of the starting substances A, I in the different solutions can vary;
that in the multiple solutions equilibrium reactions of the first equilibrium reaction A+I⇄AI take place between the starting substances, until the respective equilibrium of the first equilibrium reaction with the respective equilibrium concentrations c_{eq}(A), c_{eq}(I), c_{eq}(AI) is reached in each of the multiple solutions respectively,
that the dissolved starting substances A, I at their respective reaction equilibrium are then analyzed in any order against a ligand L for determining the equilibrium concentration of the second starting substance A, whereby at least the second starting substance A binds to ligand L according to a second equilibrium reaction, whereby the analyses are executed without any regeneration steps between exchanging the multiple solutions, whereby analysis results are obtained;
that the analysis results are evaluated in respect to their respective initial rate or other measuring values;
that via the respective initial rate or via the other measuring values an unknown concentration c_{eq}(A) of the second starting substance A in solution is determined,
that based on the determined concentration c_{eq}(A) and the starting concentrations c₀(A), c₀(I) the equilibrium concentrations of all components c_{eq}(I), c_{eq}(A), c_{eq}(AI) of the first equilibrium reaction are determined; and
that via the law of mass action (LMA) the equilibrium constant of the first equilibrium reaction is determined from the equilibrium concentrations c_{eq}(A), c_{eq}(I), c_{eq}(AI).

2. The method of claim 1, characterized in that the number of different starting substances of the preceding equilibrium reactions is two, respectively.

3. The method of claim 1, characterized in that the solutions of the different starting substances consist of mixtures of an analyte A and an inhibitor I.

4. The method of claim 1, characterized in that the other measuring values are the exponent kₒₙ or the final equilibrium state.

5. The method of claim 1, characterized in that the solutions of the different starting substances are analyzed by MSK in the order of increasing starting concentration of one of their starting substances.

6. The method of claim 1, characterized in that the MSK analysis comprises the execution of a reference measurement which is used for reference purposes, whereby solutions comprise only one of the different starting substances.

7. The method of claim 3, characterized in that the concentration of the analyte is equal to the concentration of the inhibitor.

8. The method of claim 3, characterized in that the concentration of the analyte is unequal to the concentration of the inhibitor.

9. A method for determining a first equilibrium constant of a first equilibrium reaction comprising the following steps:
Providing two or more solutions, whereby each of these solutions contains all first educts and first products participating in the first equilibrium reaction, whereby the first educts and the first products of the first equilibrium reaction are referred to as first reactants, whereby the first reactants of the respective solutions are in the reaction equilibrium of the first equilibrium reaction, whereby in the reaction equilibrium of the first equilibrium reaction the first reactants are present in their respective equilibrium concentration;
Providing a ligand, which, together with at least one of the first reactants, which is referred to as analyte, can be involved in a second equilibrium reaction, whereby the ligand and the analyte respectively act as second educts of the second equilibrium reaction;
Bringing together the two or more solutions and the ligand, so that the second equilibrium reaction takes place;
Conducting a MSK analysis of the second equilibrium reaction taking place with the two or more solutions, whereby a MSK result is obtained;
Determining the first equilibrium constant by using the MSK result.

10. The method of claim 9, whereby the number of the first educts is two and the analyte is an educt of the first equilibrium reaction.

11. The method according to claim 10, whereby one of the two first educts is an analyte and the other one of the two first educts is an inhibitor.

12. The method of claim 9,
whereby the MSK result consists of a MSK exponent kₒₙ, a MSK final equilibrium state R_{eq}, a plot comprising measured initial rates for different starting concentrations of the analyte, or an initial rate at a basis point,
whereby the basis point is indicative of a moment in a MSK analysis when the MSK analysis is started and when the ligand has not yet bound the analyte;
whereby the method further includes the steps of:
Determining the equilibrium concentration c_{eq}(A) of the analyte from the MSK result;
Calculating the first equilibrium constant from the equilibrium concentration c_{eq}(A) of the analyte by using the law of mass action.

13. The method of claim 9, whereby the MSK result is used together with a reference line in order to determine the first equilibrium constant, whereby the reference line is indicative of an initial rate in dependence on the starting concentration c₀(A) of the analyte, whereby for the dependency being described by the reference line solely the second equilibrium reaction is evaluated, not, however, the first equilibrium reaction.

14. The method of claim 13, whereby the reference line is created by executing one of the following methods:
Reading the reference line having been stored on a data storage medium;
Reading reference values having been stored on a data storage medium for automatically calculating a regression line for the read reference values, whereby the calculated regression line is used as reference line;
Performing a reference measurement with multiple additional solutions, whereby said solutions merely comprise second reactants of the second equilibrium reaction including the analyte, whereby the solutions are brought in contact with the ligand and whereby the initial rates for multiple starting concentrations of the at least one first educt are determined, and whereby a regression line is determined for the recorded reference values, whereby the reference measurement is preferentially carried out in the form of a MSK analysis.

15. The method of claim 9, whereby the ligand is immobilized.

16. The method of claim 9, whereby the MSK analysis is applied by using one of the following assay approaches: ELISA, RIA or biosensors.

17. Method for determining and visualizing the strength of a first inhibitor, whereby the first inhibitor, by binding to an analyte, inhibits the binding of the analyte to a ligand, the method comprising the following steps:
Displaying a plot on a graphical user interface of a computer system whose ordinate is indicative of measured initial rates of linearly increasing value, and whose abscissa is indicative of starting concentrations c₀(A) of the analyte of linearly increasing amount,
Plotting a reference line, whereby the reference line is indicative of the initial rates at given starting concentrations c₀(A) of the analyte;
Plotting two or more first values having been determined in a MSK analysis with two or more first solutions, whereby in said two or more first solutions the at least one analyte and the first inhibitor were dissolved, whereby each first value is indicative of an initial rate at a given starting concentration c₀(A) of the analyte in one of the two or more first solutions,
Determining a point P₁₅₀ in the plot,
whereby P₁₅₀ is one of the two or more first values or is a value on a regression curve through the two or more first values,
whereby P₁₅₀ is being determined as a point of P₁₅₀ whose ordinate value is essentially half as high as the ordinate value of the reference line at a starting concentration c₀(A) of the first analyte;
Determining the inhibitory strength of the first inhibitor as a result value which is inversely proportional to the height of the starting concentrations c₀(A) of the first analyte in Point P₁₅₀, so that the P₁₅₀ of a strong inhibitor is displayed in the plot further to the left as the P₁₅₀ of a weak inhibitor.

18. The method of claim 17, whereby the two or more first values are determined by the following steps:
Providing the two or more first solutions, whereby each of said first solutions comprises the first inhibitor and the at least one analyte, whereby the first inhibitor and the at least one analyte are involved as first educts in a first equilibrium reaction, whereby in the reaction equilibrium of the first equilibrium reaction the first educts and the first products of this first equilibrium reaction exist in their respective equilibrium concentration, whereby the concentration of the at least one analyte in the two or more first solutions is different respectively;
Providing a ligand which can be involved together with the analyte in a second equilibrium reaction, whereby the ligand and the at least one analyte respectively act as second educts of the second equilibrium reaction;
Bringing together the two or more first solutions and the ligand, so that the second equilibrium reaction takes place;
Conducting a first MSK analysis of the second equilibrium reaction taking place in the two or more first solutions for determining two or more initial rates for the two or more first solutions, whereby the initial rates are returned as first values;

19. The method of claim 18, whereby in addition the strength of the first inhibitor is compared with the strength of the second inhibitor, with the steps:
Plotting additional two or more second values having been determined in a MSK analysis with two or more second solutions, whereby in said two or more second solutions the at least one analyte and a second inhibitor were dissolved, whereby each additional second value is indicative of an initial rate at a given starting concentration c₀(A) of the at least one analyte in one of the two or more second solutions,
Determining a first regression curve for the two or more first values,
Determining a second regression curve for the two or more second values,
Determining the one inhibitor as stronger inhibitor which, at any starting concentration c₀(A) of the at least one analyte, has a lower initial rate than the other inhibitor.
